(19) United States
(12) Patent Application Publication
(10) Pub. No.: US 2025/0345211 A1
(43) Pub. Date: Nov. 13, 2025

(54) ANTI-FLUTTER EYELID COVERING SYSTEM AND METHODS OF USE

(71) Applicant: Ashley HAIGHT, Cape Coral, FL (US)

(72) Inventor: Ashley HAIGHT, Cape Coral, FL (US)

(21) Appl. No.: 19/180,062

(22) Filed: Apr. 15, 2025

Related U.S. Application Data
(60) Provisional application No. 63/644,241, filed on May 8, 2024.

(51) Int. Cl. A61F 13/12; A61F 13/02

(52) U.S. Cl. CPC A61F 13/124; A61F 13/0259

(57) ABSTRACT

An eyelid covering and metal eye covering that calms eyelid movement. It is the first available option made to artists in this field that gives them a solution to help a widely known problem with safety in the lash field. An anti-flutter eyelid covering system has an eyelid shroud shaped to cover a user's eyelid in use, and defining an exterior face and a base face; an adhesive on the base face of the eyelid shroud; in which the eyelid shroud is weighted, or has a weighted component that is weighted, to, in use, press by gravity upon the eyelid sufficiently to reduce a natural fluttering effect of a user's eyelid while the user's head is reclined.

### TECHNICAL FIELD

This document relates to anti-flutter eyelid covering systems and methods of use.

### BACKGROUND

The following paragraphs are not an admission that anything discussed in them is prior art or part of the knowledge of persons skilled in the art.

Eyelash and eyebrow extension technicians have found that involuntary eye-fluttering and opening during an eyelash extension service, eyebrow service, or lash lift service may be dangerous because it potentially allows chemicals to reach the eye. Attempts to solve this problem by others include sanitizing a coin and taping it to the eye, however, this is highly unsanitary and a completely outdated suggestion for artists in today's industry.

### SUMMARY

A professional and sanitary solution is needed. The present disclosure provides a practical and effective option for artists in this field that gives them a solution to help a common and widely known problem in the lash field.

An anti-flutter eyelid covering system is disclosed comprising: an eyelid shroud shaped to cover a user's eyelid in use, and defining an exterior face and a base face; an adhesive on the base face of the eyelid shroud; and a removable release liner covering the base face; in which the eyelid shroud is weighted, or has a weighted component that is weighted, to, in use, press by gravity upon the eyelid sufficiently to reduce a natural fluttering effect of a user's eyelid while the user's head is reclined.

A method is disclosed comprising: removing the removable release liner from the eyelid shroud of the anti-flutter eyelid covering system; and adhering the eyelid shroud to a user's eyelid.

A method is disclosed comprising: applying an eyelid shroud to cover a user's eyelid, in which an adhesive on a base face of the eyelid shroud secures the eyelid shroud to the eyelid, in which the eyelid shroud is weighted, or has a weighted component that is weighted, to, in use, press by gravity upon the eyelid sufficiently to reduce a natural fluttering effect of a user's eyelid while the user's head is reclined; and carrying out an eyelash or eyebrow enhancement treatment to eyelashes or eyebrows that border the user's eyelid while the eyelid shroud remains on the user's eyelid.

An anti-flutter eyelid covering system is disclosed comprising: an eyelid shroud shaped to cover a user's eyelid in use, and defining an exterior face and a base face; an adhesive on the base face of the eyelid shroud; in which the eyelid shroud is weighted, or has a weighted component that is weighted, to, in use, press by gravity upon the eyelid sufficiently to reduce a natural fluttering effect of a user's eyelid while the user's head is reclined.

The action of an eye fluttering and opening during an eyelash extension service, eyebrow service, or lash lift service may be dangerous as it may allow chemicals to reach the eye. The anti-flutter eyelid covering system presently disclosed keeps the eyelid closed and prevents twitching during a service to reduce possible exposure to the chemicals used during a service. The covering system may act as an eye-calming covering, keeping the eyelid closed and preventing twitching or fluttering during an eyelash extension service, which reduces possible exposure to the chemicals used during a service.

The anti-flutter eyelid covering system may use a light-blocking material to prevent light reaching the eye. Such may solve light sensitivity problems. A metal piece or substance in the middle of the covering may provide a cooling and slightly weighted piece which causes the eyelid to relax.

Various embodiments are disclosed of an anti-flutter eyelid covering system, including one that has a dark, light blocking, hypoallergenic material that adheres to the eyelid and a small area of metal or cooling substance. The darkening hypoallergenic material may adhere to the skin and contains a center area of either a metal or other skin safe substance which is used to place on the eyelid during a beauty service that involves the eyes. The eyelid shroud may be taken off of a backing or out of a package and applied directly to the eyelid when the eyes are experiencing unwanted movement, twitching, opening, or any time a calming effect is desired during a beauty service regarding the eye area for the patient's comfort, safety, and the artist's ability to complete a service safely. The skin safe and light blocking material may be a medical grade black foam tape that is combined with or adhered to the metal piece. The anti-flutter eyelid covering system is then packaged with a backing to make it easy to peel off, used, and discarded after use.

The materials used to protect the eye from the light can be switched or used in different shapes and weights. The material or metal used in the center of the patch can be switched or comprise different components. The shapes of either the eyelid shroud adhered to the eyelid can change and the shape of the cooling or weighted materials adhered to the eyelid shroud can also change in shape or weight as well. The anti-flutter eyelid covering system could be made as a disposable item or switched to be made reusable. If the eyelid shroud were made of silicon, it could be reusable. If the eyelid shroud is made of foam tape it is disposable.

In use, an enhancement artist would observe a patient or client's inability to control their eyelids and use the anti-flutter eyelid covering system to calm the eyelid and proceed with their service. Beyond using the patch for beauty services, the system may also be used daily by someone who has a problem sleeping with their eyes closed due to surgery or medical problems that don't allow the eyes to stay calmly closed.

In various embodiments, there may be included any one or more of the following features: The eyelid shroud is structured to be light blocking. The eyelid shroud comprises the weighted component. The weighted component is mounted on or embedded within the base face. The weighted component comprises metal. The weighted component has the shape of a circle. One or both the eyelid shroud or weighted component has a relatively high thermal effusivity to provide a cooling effect when in contact with a user's eyelid in use. The eyelid shroud is sufficiently flexible to conform to the three-dimensional shape of a user's eyelid, in use. The eyelid shroud comprises silicon. The eyelid shroud and adhesive are collectively formed as a foam tape. The eyelid shroud has a crescent shape. The eyelid shroud has the shape of a curvilinear trapezoid. The adhesive comprises a pressure-sensitive adhesive. The eyelid shroud is shaped to cover the user's upper eyelid. The eyelid shroud is shaped to cover the upper eyelid from a superior orbital rim to a lash margin of the user, as well as from the left to the right corners of the upper eyelid. Removing the removable release liner from the eyelid shroud of the anti-flutter eyelid covering system. Adhering the eyelid shroud to a user's eyelid. Carrying out an eyelash or brow extension process on the user while the eyelid shroud is adhered to the user's eyelid.

The foregoing summary is not intended to summarize each potential embodiment or every aspect of the subject matter of the present disclosure. These and other aspects of the device and method are set out in the claims.

### BRIEF DESCRIPTION OF THE FIGURES

Embodiments will now be described with reference to the figures, in which like reference characters denote like elements, by way of example, and in which:

FIG. 1 is a top plan exploded view of an anti-flutter eyelid covering system with an eyelid shroud, weighted component and a removable release liner, with components moved laterally relative to one another to illustrate the sequence of parts.

FIG. 2 is a side elevation exploded view of the anti-flutter eyelid covering system of FIG. 1.

FIG. 3 is a bottom plan view of the anti-flutter eyelid covering system of FIG. 1, with the removable release liner removed.

FIG. 4 is a perspective view of the anti-flutter eyelid covering system of FIG. 1, with the removable release liner in the process of being removed from the eyelid shroud.

FIG. 5 is a top plan view of the anti-flutter eyelid covering system of FIG. 1, applied to an eyelid of a user.

FIG. 6 is a top plan view of another embodiment of the anti-flutter eyelid covering system of FIG. 1.

### DETAILED DESCRIPTION

Immaterial modifications may be made to the embodiments described here without departing from what is covered by the claims.

The eyelids each comprise an upper and a lower part, and play a key role in protecting and maintaining the health of the eyes. They consist of multiple layers, including skin, muscle, connective tissue, and glands, which work together to regulate moisture and shield the eyes from debris and excessive light. The muscles of the eyelid control opening and closing, enabling blinking to spread tears evenly across the eye's surface. Specialized glands within the eyelids produce oils that help prevent tear evaporation, keeping the eyes lubricated. Additionally, the eyelids work in coordination with the tear drainage system to remove excess moisture and debris. Sensory nerves allow for reflexive blinking in response to external stimuli, further protecting the eyes from potential harm.

A human eyelid is a thin, movable fold of skin and muscle that covers and protects the eye. It has a semi-elliptical shape, conforming to the curvature of the globe, with the upper eyelid being larger and more mobile than the lower eyelid. The upper eyelid extends from the superior orbital rim to the lash margin, while the lower eyelid spans from the inferior orbital rim to its corresponding margin. Structurally, the eyelid consists of several layers, including the skin, orbicularis oculi muscle, tarsal plate, and conjunctiva. The tarsal plate, a dense connective tissue structure, provides structural integrity and houses the meibomian glands, which secrete oils to prevent tear evaporation. The levator palpebrac superioris and Müller's muscle control the movement of the upper eyelid, while the lower eyelid is mainly repositioned by the action of gravity and the orbicularis oculi muscle. The eyelid's contour and shape contribute to ocular function by distributing the tear film evenly across the cornea and facilitating blinking, which helps maintain eye moisture and remove debris.

Eyelashes are found at the borders of both the upper and lower eyelids. Eyelashes are specialized, curved hairs that emerge from the anterior margin of the eyelids, arranged in a staggered, irregular pattern. The upper eyelid typically contains 90 to 160 lashes, while the lower eyelid has 75 to 80, with upper lashes being longer (approximately 8-12 mm) and more densely packed. Each lash follicle is associated with sebaceous (Zeis) and apocrine (Moll) glands, which secrete oils that help prevent lash brittleness and protect against microbial infections. Eyelashes function as a protective barrier, acting as highly sensitive tactile sensors that trigger the blink reflex when foreign particles or airflow are detected. Unlike scalp hair, eyelashes have a shorter, non-cyclic growth phase (anagen), lasting about 30-45 days, followed by a brief catagen phase and a prolonged telogen phase of approximately 100 days before shedding. Their curvature and upward or downward orientation prevent debris and sweat from directly entering the ocular surface, aiding in eye protection and reducing the risk of corneal irritation.

More recently, individuals have been opting to apply eyelash extensions to natural eyelashes, to enhance the length, volume, and curl of their natural lashes, achieving a more defined and aesthetically appealing eye appearance without the need for daily mascara or lash curling. Eyelash extensions are semi-permanent fibers individually applied to natural lashes using a medical-grade adhesive, offering a customized look based on desired thickness, length, and curl type. They provide a time-saving solution for individuals seeking a low-maintenance beauty routine while maintaining a polished appearance. Extensions are particularly beneficial for those with sparse, short, or straight lashes, as they create a fuller and more lifted effect. Additionally, they can serve functional purposes, such as reducing reliance on makeup products that may cause irritation or allergic reactions, especially for individuals with sensitive eyes or contact lens wearers. Eyelash extensions also offer a long-lasting alternative to strip lashes, remaining intact for several weeks with proper care, making them ideal for special occasions, professional settings, or daily wear.

Eyelash extensions are often synthetic, silk, or mink fibers individually attached to natural eyelashes using a semi-permanent adhesive to enhance length, volume, and curl. The application process requires precision and typically begins with cleansing the natural lashes to remove oils and debris, ensuring proper adhesion. A protective gel pad or medical tape may be placed on the lower eyelids to isolate the upper lashes. Using fine-tipped tweezers, a trained lash artist selects and isolates a single natural lash before dipping the extension in an adhesive, typically a cyanoacrylate-based adhesive, and carefully affixing it approximately 1-2 mm from the eyelid. This process is repeated for each lash, with consideration for weight and length to maintain the health of the natural lashes. The adhesive cures within minutes, but full bonding strength is achieved over 24 hours. Proper aftercare, including avoiding moisture and oil-based products, helps extend the longevity of the extensions, which naturally shed with the lash growth cycle over 4-6 weeks.

Eyebrows are arched structures composed of dense, terminal hairs positioned along the supraorbital ridge, playing a crucial role in facial expression, eye protection, and aesthetics. Structurally, the eyebrow consists of a skin layer, hair follicles, sebaceous glands, and an underlying musculature that includes the frontalis, corrugator supercilii, and orbicularis oculi muscles, which control movement for expressive functions. The typical eyebrow shape follows a gentle curve, with a medial head, an arch that peaks near the lateral third, and a tapering tail. Functionally, eyebrows deflect sweat, debris, and moisture away from the eyes while enhancing nonverbal communication by conveying emotions such as surprise, concern, or anger. Hair growth follows a unique cycle with a longer anagen phase compared to eyelashes but shorter than scalp hair, contributing to their relatively stable length. The density, thickness, and shape of eyebrows vary based on genetic, hormonal, and environmental factors, influencing their role in facial symmetry and perception. Eyebrow grooming, including shaping, tinting, microblading, and lamination, is commonly performed for both cosmetic and reconstructive purposes to enhance facial harmony and restore lost or sparse hair.

An individual may seek to have eyebrow extensions or enhancements applied to improve the fullness, shape, and definition of their brows, particularly if they have sparse, thinning, or asymmetrical eyebrows due to genetics, aging, over-plucking, medical conditions, or hair loss disorders such as alopecia. These enhancements provide a non-invasive or semi-permanent solution for achieving a more polished and symmetrical brow appearance. There are several types of eyebrow enhancement procedures available. Eyebrow extensions involve attaching synthetic or natural hair fibers to existing brow hairs or directly onto the skin using medical-grade adhesive, offering a temporary, natural-looking boost lasting up to two weeks. Microblading is a semi-permanent tattooing technique in which fine, hair-like strokes are etched into the skin using a manual blade and pigment, creating a realistic brow effect that lasts 1-3 years. Powder brows (shading or ombre brows) use a machine to deposit pigment in a soft, gradient effect, mimicking the look of filled-in makeup and lasting up to three years. Nano brows, similar to microblading but performed with an ultra-fine tattoo needle, provide even more precise, delicate strokes with longer-lasting results. Brow lamination is a non-invasive treatment that relaxes and reshapes natural eyebrow hairs using a chemical solution, creating a lifted, fuller appearance that lasts 6-8 weeks. These procedures cater to different aesthetic preferences, from a soft, natural enhancement to a bold, defined brow, allowing individuals to achieve a customized look that complements their facial features.

Eyebrow extensions include individual synthetic or natural hair fibers applied to the existing brow hairs or directly onto the skin using a medical-grade adhesive to enhance fullness, shape, and definition. The application process begins with cleansing the brow area to remove oils and debris, ensuring optimal adhesion. A trained technician maps the desired brow shape based on facial symmetry and natural brow structure. If the client has existing brow hairs, individual extensions are attached to them using a fine-tipped applicator and adhesive. For clients with sparse or no natural brows, extensions are adhered directly to the skin using a specialized bonding technique. The process requires precision to ensure natural-looking results, with considerations for hair length, thickness, and direction of growth. The adhesive fully cures within 24 hours, and proper aftercare, including avoiding excessive moisture and oil-based products, helps maintain longevity. Eyebrow extensions typically last up to two weeks before requiring touch-ups due to natural shedding and skin exfoliation.

While eyelash and eyebrow extensions can enhance aesthetics, they also pose potential risks and complications. One primary concern is allergic reactions to the adhesives used, which may contain cyanoacrylate or latex, leading to redness, swelling, and irritation. Contact dermatitis and blepharitis (inflammation of the eyelid margin) can develop due to sensitivity to the glue or improper hygiene. Follicular damage is another risk, as repeated application of extensions can stress hair follicles, leading to traction alopecia, where natural lashes or brows become weaker, sparser, or stop growing altogether. Poor application techniques can also cause lash clumping, which puts excessive strain on natural lashes and increases the likelihood of premature shedding. In the case of eyebrow enhancements like microblading, improper technique or low-quality pigments may result in scarring, uneven pigmentation, or color fading to unnatural hues over time. Additionally, bacterial and fungal infections can occur if tools and materials are not properly sanitized, particularly in semi-permanent procedures that break the skin barrier. To minimize risks, extensions are typically applied by trained professionals using high-quality, hypoallergenic materials, and proper aftercare must be followed to maintain hygiene and prevent complications.

The eyelashes have a tendency to flutter, at various times of the day and in different situations. For example, the eyelashes will experience a natural eyelash fluttering effect as a result of rapid, involuntary or voluntary movement of the eyelids, creating a delicate, rhythmic motion of the eyelashes. This effect is primarily driven by the coordinated action of the orbicularis oculi and levator palpebrae superioris muscles, which control eyelid closure and opening, respectively. Subtle micro-movements, influenced by neural signals from the facial and oculomotor nerves, contribute to the fluttering motion, often observed during rapid eye movement (REM) sleep, emotional responses, or intentional blinking patterns. The soft, feathery movement of the lashes enhances visual appeal, as light catches the fine hair fibers, creating a subtle dynamic effect. Factors such as lash length, curl, and density can amplify the fluttering effect, making it more pronounced. Additionally, external elements like air movement or blinking speed can further influence how the eyelashes flutter, adding to their natural aesthetic and functional role in protecting the eyes.

Involuntary eyelash fluttering is commonly experienced during an eyelash or eyebrow treatment, and can pose complications for the technician performing the treatment and the individual receiving the treatment. Involuntary eyelash fluttering may occur in response to objects in close proximity to the eye, and is a protective reflex primarily governed by the corneal reflex arc and the blink reflex. This reaction may be triggered when mechanoreceptors in the cornea or eyelid margin detect subtle changes in airflow, pressure, or the presence of a foreign object, such as a finger, debris, or sudden movement near the eye. The sensory input is transmitted via the ophthalmic branch (V1) of the trigeminal nerve (CN V) to the brainstem's trigeminal nucleus, which then rapidly signals the facial nerve (CN VII) to activate the orbicularis oculi muscle, causing the eyelids to blink or flutter. This rapid, repetitive movement helps disperse the tear film, remove potential irritants, and shield the ocular surface from injury. Eyelash fluttering can also be exacerbated by heightened neuromuscular sensitivity, stress, or fatigue of the oculomotor system, leading to increased responsiveness to external stimuli. Additionally, enhanced peripheral vision sensitivity and subconscious anticipation of contact may contribute to the phenomenon, reinforcing the eye's natural defense mechanism.

Involuntary eyelash fluttering during extension services poses significant challenges and risks, primarily due to the precision required when applying cyanoacrylate-based or other adhesives. These fast-drying adhesives release trace amounts of formaldehyde as they cure, which can cause irritation if the eyes are not fully closed. Excessive eyelid movement during application can lead to improper adhesion, clumping, or misalignment of extensions, compromising both aesthetics and lash health. Additionally, fluttering increases the risk of adhesive fumes entering the eye, potentially leading to chemical burns, redness, or watery eyes. Factors such as heightened sensitivity, anxiety, or underlying neurological conditions can contribute to involuntary eyelid fluttering. Many lash technicians assess a client's ability to remain still before proceeding with the service, and persistent fluttering may result in service refusal to prevent injury and ensure optimal results. To mitigate risks, some artists use sensitive-formula adhesives with lower fumes or employ taping techniques to stabilize the eyelid, but extreme cases may still be unsuitable for the procedure.

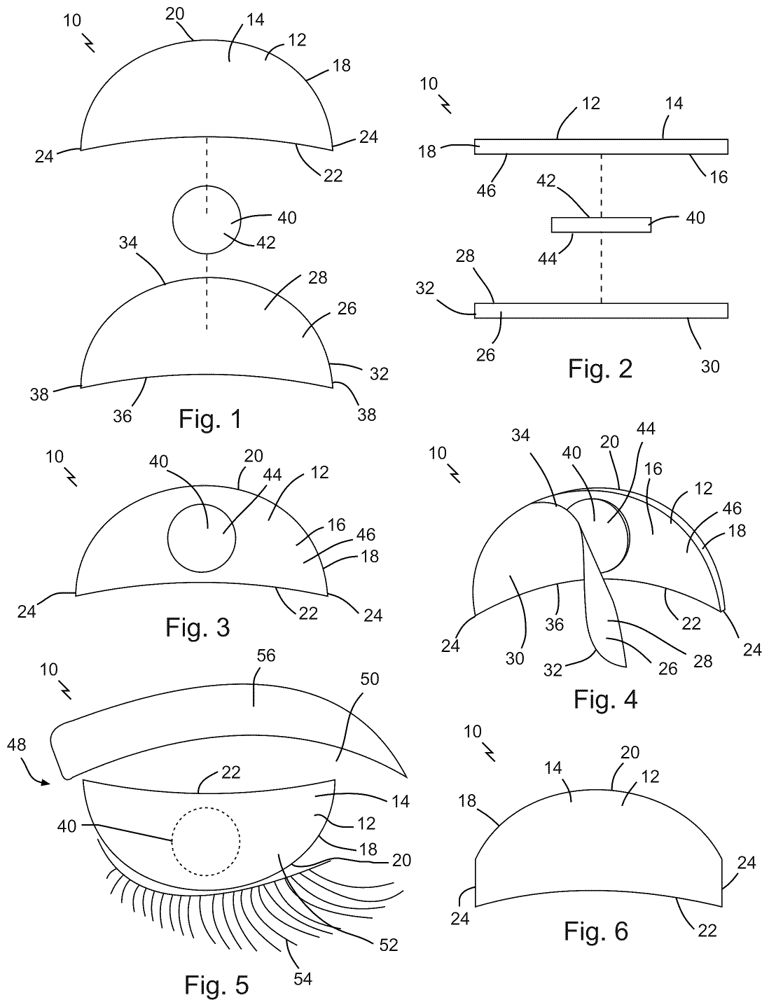

Referring to FIGS. 1-5, an anti-flutter eyelid covering system 10 is disclosed. The system 10 comprises an eyelid shroud 12. The system 10 may comprise a removable release liner 26. The eyelid shroud 12 may be shaped to cover a user's eyelid in use. The eyelid shroud 12 may define an exterior face 14 and a base face 16. An adhesive, such as an adhesive coating 46, may be applied to or otherwise active upon the base face 16 of the eyelid shroud 12. The adhesive may be protected prior to use by being covered by a removable release liner 26. The material itself of the eyelid shroud 12 may inherently comprise adhesive properties, rather than simply being coated by an adhesive. The eyelid shroud 12 may be weighted, so that, in use, the eyelid shroud 12 presses by gravity upon the eyelid 52 sufficiently to reduce a natural fluttering effect of a user's eyelid 52, while the user's head is reclined, to allow the shroud 12 to rest upon the eyelid. A weighted component 40, such as a disc, may provide the weight to the eyelid shroud 12. The anti-flutter eyelid covering system 10 may be applied to an eye area 48 of a user 50 by removing the removable release liner 26 from the eyelid shroud 12 and adhering the eyelid shroud 12 to the user's eyelid 52. Once the anti-fluttering eyelid covering system 10 is applied an eyelash or brow extension process may be carried out on the user while the eyelid shroud 12 is adhered to the user's eyelid 52.

Light exposure to the eye may be a key factor in causing involuntary eyelash fluttering. Light plays a crucial role in influencing eyelid behavior and the eyelash fluttering effect, as the eyelids function as a protective barrier regulating the amount of light that reaches the eyes. Sudden exposure to bright light can trigger an involuntary blinking or fluttering response due to the photic reflex, mediated by the optic nerve and oculomotor pathways. This reflex helps protect the retina from excessive light exposure and prevents discomfort. In addition, exposure to light, such as from a lamp used by a lash technician, may increase eyelid sensitivity to fluttering even when the eyelid, which is translucent, is closed. In darker environments, reduced light stimuli may allow the eyelids to relax, minimizing fluttering. Artificial lighting used during eyelash extension procedures, such as LED or fluorescent lamps, can sometimes induce subtle eyelid movements due to glare or visual strain. The intensity and wavelength of light may impact individuals with light sensitivity, leading to increased fluttering, especially in those with underlying neurological conditions. As a result of the above, lash technicians may have to adjust lighting angles or use lower-intensity illumination to minimize unnecessary eye movement and ensure precise application during eyelash extension services.

Referring to FIGS. 1-5, the eyelid shroud 12 may comprise light-blocking characteristics. The light-blocking characteristics of the eyelid shroud 12 may reduce or eliminate eyelash fluttering that is caused by exposure to light. Light-blocking materials may function by absorbing, reflecting, or diffusing incoming light to prevent transmission through the material. The effectiveness of a material's light-blocking capability may be determined by its opacity, thickness, composition, and surface treatment. Key factors include high-density fiber structures, metallic or polymeric coatings, and pigment concentration that enhance absorption or reflection. Materials such as blackout fabrics, tinted films, and specialized coatings incorporate carbon black, aluminum layers, or UV-resistant additives to optimize light-blocking performance. The degree of light blockage is quantified using optical density (OD), transmittance percentage, and shading coefficient. Optical density (OD), measured on a logarithmic scale, indicates how much light is attenuated (e.g., OD 3 blocks 99.9% of light). Total transmittance (T %), calculated using spectrophotometry, represents the percentage of incident light passing through the material, where lower values indicate stronger blockage. Additionally, the shading coefficient (SC), commonly used in window films, assesses the material's ability to reduce solar heat gain. These metrics allow for standardized evaluation of light-blocking efficiency across applications in architecture, optical devices, textiles, and industrial shielding.

Referring to FIGS. 1-5, the eyelid shroud 12 may comprise a suitable weighted component 40. Applying a weight to the eyelid may help reduce involuntary eyelash fluttering by increasing mechanical resistance and stabilizing the eyelid's neuromuscular response to stimuli. Similar to how weighted blankets (typically 7-12% of body weight) exert deep pressure stimulation (DPS) to reduce anxiety and involuntary movement, a weighted eyelid component may provide gentle, consistent pressure to modulate the reflexive blinking response. The weight may need to be carefully calibrated to avoid excessive strain while still damping the rapid contractions of the orbicularis oculi muscle. Studies on gold or platinum eyelid implants for patients with lagophthalmos suggest that weights of 1.0-1.8 grams are sufficient to influence eyelid mechanics without discomfort. In some cases, the weight of the weighted component 40 may range from 0.5-3.0 grams. By comparison, a non-weighted, covered eyelid may experience unopposed muscle contractions, leading to heightened sensitivity to external stimuli such as airflow or proximity detection. The application of a lightweight material-such as a thin gold, metal alloys, cupronickel, bronze, brass, nickel, steel, zinc, aluminum, silicone, or polymer-based components may suppress excessive fluttering by subtly increasing the force required for involuntary movement, thus promoting a more controlled blinking response. The extent of weighting may be selected for the user to provide an optimal mass-to-surface area ratio for effective suppression without impeding voluntary eyelid motion.

Referring to FIGS. 1-5, in some cases, a weighted component 40 is used distinct from the eyelid shroud itself. The weighted component 40 may have suitable characteristics. The weighted be mounted on or embedded within the base face 16 of the eyelid shroud 12. If embedded, the weighted component may be embedded fully or partially within a confirming slot in the base face or may be fully encased by the structure of the shroud so as not to come in direct contact with the user in use. The weighted component 40 may comprise a shroud contacting face 42 and a base face 44. The base face 44 may contact the eyelid 52 of the user 50 during use. The weighted component 40 may comprise metal, or the other materials discussed elsewhere in this paragraph. The weighted component 40 may have a suitable shape, for example a circle, although other shapes may be used. The weighted component or weighting may be distributed about the surface area of the shroud 12, or centered such as when the weighted component 40 is in the center of the shroud as shown.

Referring to FIGS. 1-5, one or both of the eyelid shroud 12 or weighted component 40 may be structured to have a relatively high thermal effusivity to provide a cooling effect when in contact with a user's eyelid 52 in use. An object feels cool to the touch due to its thermal effusivity, which determines how quickly it can exchange heat with another material, such as human skin. Thermal effusivity is a property influenced by both thermal conductivity, the rate at which heat moves through a material, and heat capacity, the amount of heat a material can store. Materials with high thermal effusivity, such as metals, may rapidly absorb heat from the skin, creating a cool sensation. A cooling sensation may act to further calm the natural reflex of the eyelid to stimuli, calming the eyelid and reducing involuntary fluttering. In contrast, materials with low thermal effusivity, like wood or fabric, transfer heat more slowly and feel warmer. Surface characteristics also affect perceived coolness, as smooth surfaces enhance heat transfer, while rough or porous materials reduce direct contact and slow down heat exchange. One or both of the eyelid shroud 12 and the weighted component 40 may comprise characteristics that provide them with relatively high thermal effusivity. Thermal effusivity (eee), measured in J/(m²·K·s^{0.5}), quantifies a material's ability to exchange heat with its surroundings and is defined by the equation:

$$e = \sqrt{k ρ {c}_{p}}$$

where k is thermal conductivity (W/m·K), ρ is density (kg/m³), and cp is specific heat capacity (J/kg·K). High thermal effusivity materials (>10,000 J/m²·K·s^{0.5}), such as copper (˜40,000 J/m²·K·s^{0.5}), aluminum (˜24,000 J/m²·K·s^{0.5}), and steel (˜16,000 J/m²·K·s^{0.5}), quickly absorb and dissipate heat, making them feel cold to the touch. Low thermal effusivity materials (<1,000 J/m²·K·s^{0.5}), such as air (˜6 J/m²·K·s^{0.5}), polystyrene foam (˜50 J/m²·K·s^{0.5}), wood (˜500 J/m²·K·s^{0.5}), and human skin (˜500 J/m²·K·s^{0.5}), resist heat transfer, making them feel warm upon contact. This property is critical in designing thermal insulation, textiles, and materials for human interaction, where comfort and energy efficiency are key considerations.

Referring to FIGS. 1-6, the eyelid shroud 12 may be sufficiently flexible to conform to the three-dimensional shape of a user's eyelid 52, such as the upper eyelid, in use. The eyelid shroud 12 may be sufficiently resilient to maintain the shape of the eyelid shroud 12 when it is not in use. Once the eyelid shroud 12 has been applied to the user's eyelid 52 by adhesion, the shroud 12 may conform to the user's eyelid 52. The eyelid shroud may be shaped to cover the upper eyelid, for example from a superior orbital rim to a lash margin of the user, as well as from the left to the right corners of the upper eyelid. For an object or material to conform to the shape of the eyelid, it must exhibit flexibility, adaptability, and surface compliance to align with the natural curvature and movement of the eyelid. The eyelid is a thin, pliable structure that follows the convex contour of the globe of the eye and consists of key anatomical components, including the tarsal plate (dense connective tissue providing structural support), orbicularis oculi muscle (responsible for eyelid closure), palpebral conjunctiva (inner mucosal lining), and skin (one of the thinnest in the human body, with minimal subcutaneous fat). Materials intended to conform to the eyelid-such as ocular prosthetics, eyelid weights, or adhesive patches-must accommodate both static curvature (when the eyelid is at rest) and dynamic movement (blinking and closing). The degree of conformity is influenced by material elasticity, thickness, and adhesive properties, ensuring a secure fit without excessive pressure that could obstruct the meibomian glands (responsible for tear film stability) or interfere with the levator palpebrae superioris muscle (which controls eyelid elevation). Proper conformation enhances comfort, functionality, and therapeutic efficacy in medical or cosmetic applications, reducing irritation and promoting long-term wearability.

Referring to FIGS. 1-6, the anti-fluttering eyelid covering system 10 or eyelid shroud 12 may comprise suitable materials. The anti-fluttering eyelid covering system 10 may be disposable, for example, the system 10 may comprise a foam tape, in a peel-and stick configuration. The eyelid shroud 12 and the adhesive coating 46 may collectively be formed by the foam tape. The anti-fluttering eyelid covering system 10 may be structured to be reusable, for example, the system 10 may comprise silicon, such as a silicon pad. The eyelid shroud 12 itself may comprise silicon. In the example where the system 10 is reusable, the adhesive coating 46 may be reusable or may not be needed. The silicon eye shroud 12 may provide enough friction with the user's eyelid 52 to secure the system 10 to the eye area 48 of the user 50. When selecting alternative materials to silicone for direct skin contact, factors such as biocompatibility, flexibility, durability, and hypoallergenic properties must be considered. Thermoplastic elastomers (TPEs), such as styrene-based elastomers (SBS, SEBS) and polyurethane (TPU), offer softness, elasticity, and skin safety, making them suitable for wearable medical devices and prosthetics. Fluoroelastomers (FKM), like Viton, provide high chemical resistance and durability but are less commonly used for prolonged skin contact due to their stiffness. Medical-grade polyurethanes are widely used in wound dressings and wearable electronics due to their breathability and moisture permeability. Hydrogels, composed of crosslinked polymer networks (e.g., polyvinyl alcohol or polyethylene glycol), offer soft, moisture-retaining properties, making them ideal for adhesive patches, prosthetics, and transdermal drug delivery systems. Ethylene vinyl acetate (EVA) is a flexible, lightweight material used in orthotics and wearable pads, providing shock absorption and comfort. Additionally, textile-based composites incorporating coated fabrics, bioengineered silk, or graphene-infused fibers can provide adaptive, skin-friendly surfaces for long-term applications. These alternatives are designed to mimic or exceed silicone's mechanical properties while addressing concerns such as allergic reactions, oil absorption, or environmental impact associated with silicone use.

Referring to FIGS. 1-5, the eyelid shroud 12 may comprise a suitable shape. For example, the eyelid shroud 12 may comprise a crescent shape. The eyelid shroud 12 may define a peripheral edge 18. The peripheral edge 18 may comprise a leading edge 20 and a trailing edge 22, which may join to form opposed tips 24. The leading edge 20 may comprise a relatively higher degree of curvature than the trailing edge 22. In use, the leading edge 20 may be placed adjacent to, for example bordering the eyelashes 54 while the trailing edge 22 may be placed next to the eyebrow 56. In the same example, the eyelid shroud 12 may be referred to as having the shape of a curvilinear trapezoid. In the alternative shown in FIG. 6, the shape may be modified, for example to include straight tips 24 between elliptical sections of the leading and trailing edges.

Referring to FIGS. 2-4, a suitable adhesive coating 46 may be used. The adhesive coating 46 may comprise a pressure-sensitive adhesive. A lash technician may lightly press the eyelid shroud 12 onto the eyelid 52 of a user 50, which may activate the pressure coating 46 and secure the eyelid shroud 12. Pressure-sensitive adhesives (PSAs) are viscoelastic materials that form a bond with a substrate upon the application of light pressure, without requiring heat, water, or solvents. These adhesives rely on tack, cohesion, and adhesion properties to maintain effective contact and attachment. The composition of PSAs typically includes elastomers (e.g., acrylics, silicones, or rubber-based polymers), tackifiers (resins that enhance stickiness), plasticizers (to improve flexibility), and stabilizers (for durability and resistance to environmental factors). Acrylic-based PSAs offer UV resistance and durability, making them ideal for medical tapes and labels, while silicone-based PSAs provide high-temperature resistance and biocompatibility, suitable for skin-contact applications such as medical electrodes and wearable sensors. Rubber-based PSAs have high initial tack and flexibility but degrade faster in extreme conditions. The performance of a PSA is quantified by shear strength (resistance to sliding), peel strength (force required to remove it from a surface), and tack (instant adhesion on contact). These adhesives are used in medical devices, automotive applications, electronics, and industrial tapes, where case of application, removability, and adaptability to various surfaces are essential.

Adhesives suitable for direct skin contact may need to be biocompatible, non-irritating, flexible, and breathable, while also providing adequate adhesion without causing skin damage upon removal. The primary types of skin-safe adhesives include pressure-sensitive adhesives (PSAs), silicone adhesives, hydrocolloid adhesives, hydrogel adhesives, and medical-grade acrylics. Silicone adhesives are widely used in medical tapes, dressings, and wearable devices due to their gentle adhesion, flexibility, and ability to conform to skin movement, making them ideal for sensitive or fragile skin. Hydrocolloid adhesives, made from gel-forming agents such as pectin or carboxymethylcellulose, provide moisture-retaining properties and are commonly used in wound care and ostomy appliances. Hydrogel adhesives, composed of water-rich, crosslinked polymer networks, offer cooling effects and gentle adhesion, making them ideal for transdermal patches, electrode pads, and burn dressings. Medical-grade acrylic adhesives provide strong, long-term adhesion with high breathability, often used in surgical drapes, medical tapes, and fixation devices. The selection of an appropriate adhesive depends on skin sensitivity, duration of wear, flexibility, moisture resistance, and removability to ensure both secure attachment and patient comfort.

Referring to FIGS. 1-5, the removable release liner 26 may have suitable properties. Release liners may serve an important role as components in peel-and-stick applications, serving as a protective barrier that covers adhesives before use, preventing premature adhesion and contamination. These liners may be made from paper, plastic films (e.g., polyethylene, polyester, or polypropylene), or composite materials and are coated with a low-surface-energy release agent, such as silicone, fluoropolymers, or wax, to facilitate easy removal. The release force—the amount of force required to separate the liner from the adhesive—may be carefully engineered to balance secure adhesive protection with effortless peeling. Silicone-coated release liners are widely used due to their consistent release performance, thermal stability, and compatibility with various adhesives (e.g., pressure-sensitive adhesives, hydrocolloid adhesives, and medical-grade acrylics). In medical and skin-contact applications, release liners must also be biocompatible, free of harmful residues, and designed to maintain sterility, often sealed in protective pouches to prevent contamination. Advanced release liners may feature differential coatings, where each side has a different release strength, or microtextured surfaces to improve handling and prevent static buildup. These liners play a crucial role in ensuring adhesive integrity, case of application, and product longevity across industries such as medical dressings, wearable sensors, wound care, and transdermal patches.

Referring to FIGS. 1-6, the eyelid shroud 12 may be provided in a hypoallergenic form. The hypoallergenic material of the eyelid shroud 12 may be selected to reduce or eliminate irritation of the user's eyelid 52 when the eyelid shroud 12 is placed on the eyelid 52 for an extended period of time. Ensuring that a skin-contacting component is hypoallergenic is critical for preventing allergic reactions, irritation, and long-term dermatological issues, particularly for individuals with sensitive skin or preexisting conditions such as eczema or contact dermatitis. This can be achieved by selecting biocompatible, dermatologically tested materials that are free from common allergens such as latex, certain adhesives, and chemical sensitizers (e.g., formaldehyde, phthalates, or parabens). Materials such as medical-grade silicones, hydrogels, and hypoallergenic acrylic adhesives are commonly used due to their low reactivity, breathability, and minimal residue upon removal. To maintain hypoallergenic properties, the component must be manufactured and stored in a contamination-free environment, avoiding exposure to potential allergens or environmental pollutants. Packaging the component in a sterile, protective sealed pouch before use helps prevent contamination, preserves material integrity, and ensures that the product remains free from bacteria, dust, and external irritants. Additionally, single-use or properly sanitized reusable designs further reduce the risk of cross-contamination. Regulatory compliance with medical and dermatological safety standards (such as ISO 10993 for biocompatibility or FDA guidelines for skin-contacting materials) is essential to confirm long-term safety and reliability in medical, cosmetic, or wearable applications.

Referring to FIGS. 4 and 5, a method of use of the eyelid covering system 10 is illustrated. The removable release liner 26 may be removed from the eyelid shroud 12. The eyelid shroud 12 may be adhered to a user's eyelid, such as the upper eyelid 52 as shown. A technician may thereafter carry out an eyelash or brow extension process on the user's eyelashes 54 or eyebrows 56, respectively, while the eyelid shroud 12 is adhered to the user's eyelid 52. After treatment, the shroud 12 may be gently peeled off the user's eyelid, and disposed of, or cleaned for re-use as the case may be.

In the claims, the word “comprising” is used in its inclusive sense and does not exclude other elements being present. The indefinite articles “a” and “an” before a claim feature do not exclude more than one of the feature being present. Each one of the individual features described here may be used in one or more embodiments and is not, by virtue only of being described here, to be construed as essential to all embodiments as defined by the claims.

The embodiments of the invention in which an exclusive property or privilege is claimed are defined as follows:

1. An anti-flutter eyelid covering system comprising:
an eyelid shroud shaped to cover a user's eyelid in use, and defining an exterior face and a base face;
an adhesive on the base face of the eyelid shroud; and
a removable release liner covering the base face;
in which the eyelid shroud is weighted, or has a weighted component that is weighted, to, in use, press by gravity upon the eyelid sufficiently to reduce a natural fluttering effect of a user's eyelid while the user's head is reclined.

2. The anti-flutter eyelid covering system of claim 1 in which the eyelid shroud is structured to be light blocking.

3. The anti-flutter eyelid covering system of claim 1 in which the eyelid shroud comprises the weighted component.

4. The anti-flutter eyelid covering system of claim 3 in which the weighted component is mounted on or embedded within the base face.

5. The anti-flutter eyelid covering system of claim 3 in which the weighted component comprises metal.

6. The anti-flutter eyelid covering system of claim 3 in which the weighted component has the shape of a circle.

7. The anti-flutter eyelid covering system of claim 1 in which one or both the eyelid shroud or weighted component has a relatively high thermal effusivity to provide a cooling effect when in contact with a user's eyelid in use.

8. The anti-flutter eyelid covering system of claim 1 in which the eyelid shroud is sufficiently flexible to conform to the three-dimensional shape of a user's eyelid, in use.

9. The anti-flutter eyelid covering system of claim 1 in which the eyelid shroud comprises silicon.

10. The anti-flutter eyelid covering system of claim 1 in which the eyelid shroud and adhesive are collectively formed as a foam tape.

11. The anti-flutter eyelid covering system of claim 1 in which the eyelid shroud has a crescent shape.

12. The anti-flutter eyelid covering system of claim 1 in which the eyelid shroud has the shape of a curvilinear trapezoid.

13. The anti-flutter eyelid covering system of claim 1 in which the adhesive comprises a pressure-sensitive adhesive.

14. The anti-flutter eyelid covering system of claim 1 in which the eyelid shroud is shaped to cover the user's upper eyelid.

15. The anti-flutter eyelid covering system of claim 14 in which the eyelid shroud is shaped to cover the upper eyelid from a superior orbital rim to a lash margin of the user, as well as from the left to the right corners of the upper eyelid.

16. A method comprising:
removing the removable release liner from the eyelid shroud of the anti-flutter eyelid covering system of claim 1; and
adhering the eyelid shroud to a user's eyelid.

17. The method of claim 16 further comprising carrying out an eyelash or brow extension process on the user while the eyelid shroud is adhered to the user's eyelid.

18. A method comprising:
applying an eyelid shroud to cover a user's eyelid, in which an adhesive on a base face of the eyelid shroud secures the eyelid shroud to the eyelid, in which the eyelid shroud is weighted, or has a weighted component that is weighted, to, in use, press by gravity upon the eyelid sufficiently to reduce a natural fluttering effect of a user's eyelid while the user's head is reclined; and
carrying out an eyelash or eyebrow enhancement treatment to eyelashes or eyebrows that border the user's eyelid while the eyelid shroud remains on the user's eyelid.
